(19) United States
(12) Patent Application Publication
(10) Pub. No.: US 2016/0289173 A1
(43) Pub. Date: Oct. 6, 2016

(54) DEVICES AND METHODS FOR CRYSTALLIZING A COMPOUND

(71) Applicant: MASSACHUSETTS INSTITUTE OF TECHNOLOGY, CAMBRIDGE, MA (US)

(72) Inventors: ALLAN STUART MYERSON, BOSTON, MA (US); RICHARD DEAN BRAATZ, ARLINGTON, MA (US); STEVEN THOMAS FERGUSON, CAMBRIDGE, MA (US); MIN SU, BEICHEN (CN); BERNHARDT LEVY TROUT, LEXINGTON, MA (US); LIFANG ZHOU, CAMBRIDGE, MA (US); NIMA YAZDAN PANAH, CAMBRIDGE, MA (US)

(73) Assignee: MASSACHUSETTS INSTITUTE OF TECHNOLOGY, CAMBRIDGE, MA (US)

(21) Appl. No.: 15/085,798

(22) Filed: Mar. 30, 2016

Related U.S. Application Data
(60) Provisional application No. 62/142,364, filed on Apr. 2, 2015.

(51) Int. Cl. C07C 231/24; C07C 51/43; B01D 9/00; C07C 67/52

(52) U.S. Cl. CPC C07C 231/24; C07C 67/52; C07C 51/43; B01D 9/0013; B01D 2009/0086; B01D 2221/10

(57) ABSTRACT

The present invention generally relates to devices and methods for crystallizing a compound. In certain industries, crystallization techniques require additional filtration steps in order to obtain products of relatively high yield and/or high purity. In some embodiments, the devices and methods described herein facilitate continuous production of high yield and/or high purity products without the need for additional filtration steps. In some embodiments, the devices and methods comprise flowing a fluid comprising a compound (e.g., a crystallizable compound, a solidifiable compound) over a substrate such that the compound crystallizes and/or precipitates on the substrate. In some embodiments, the crystallized compound can be recovered (e.g., at a high purity in solution). In certain embodiments, the substrate is orientated substantially vertically (e.g., such that flow of the fluid is driven by gravity). In some cases, the substrate comprises a plurality of crystallization promoting structures.

### RELATED APPLICATIONS

This application claims priority under 35 U.S.C. §119(e) to U.S. Provisional Application Ser. No. 62/142,364, entitled “DEVICES AND METHODS FOR CRYSTALLIZING A COMPOUND” filed on Apr. 2, 2015, which is herein incorporated by reference in its entirety.

### FIELD OF THE INVENTION

The present invention generally relates to devices and methods for crystallizing a compound.

### BACKGROUND

Crystallization is an important separation and purification process in the manufacturing of specialty chemicals, food, cosmetics, and pharmaceuticals. Batch crystallizers and continuous mixed-suspension mixed-product-removal (MSMPR) crystallizers typically require filtration in order to obtain a final purified product. While filtration is generally a major part of the crystallization process for most industries, poor filtering of crystals can result in bottlenecks in the downstream processing and may add hours or even days to the process time, which can cause significant delays and affect crystal product purity and/or reduce yield due to a need for additional washing steps.

Accordingly, improved devices and methods are needed.

### SUMMARY OF THE INVENTION

The present invention generally relates to devices and methods for crystallizing a compound.

In one aspect, methods for obtaining a crystallized compound are provided. In some embodiments, the method comprises flowing a fluid comprising the compound, the fluid having a first temperature less than the melt temperature of the compound, over at least a portion of a substrate having a second temperature less than the first temperature, such that the compound crystallizes in a crystal layer on at least a portion of the substrate, wherein the substrate is oriented substantially vertically, and recovering the crystallized compound.

In another aspect, methods for separating a solidifable compound are provided. In some embodiments, the method comprises flowing a fluid comprising the compound, the fluid having a first temperature less than the melt temperature of the compound, over at least a portion of a substrate having a second temperature less than the first temperature, such that the compound precipitates a solid on at least a portion of the substrate, wherein the substrate is oriented substantially vertically, and recovering the precipitated solid.

In yet another aspect, devices for crystallizing and/or separating a compound are provided. In some embodiments, the device comprises a substantially vertical substrate having a first portion adapted to receive a flowing fluid comprising a crystallizable compound, and a second portion adapted to receive a temperature controlling fluid, and a plurality of crystallization-promoting structures on the first portion of the substrate.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

FIGS. 1A-1D are schematic diagrams of a device for crystallizing a compound, according to one set of embodiments;

FIG. 2 is a schematic diagram of a device for crystallizing a compound, according to another set of embodiments;

FIG. 3 is a schematic diagram of a device for crystallizing a compound, according to yet another set of embodiments;

FIG. 4 is a process flow diagram of the falling film crystallizer in a recycle loop, according to one set of embodiments;

FIG. 5 is a schematic diagram of an exemplary device for crystallizing a compound, according to one embodiments; and

FIG. 6 is a photograph of a substrate comprising micropitches, according to one set of embodiments.

FIG. 7 is a photograph of a substrate comprising a crystal layer, according to one set of embodiments.

Other aspects, embodiments, and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

The present invention generally relates to devices and methods for crystallizing a compound. In certain industries, crystallization techniques require additional filtration steps in order to obtain products of relatively high yield and/or high purity. In some embodiments, the devices and methods described herein facilitate continuous production of high yield and/or high purity products without the need for additional filtration steps.

In some embodiments, the devices and methods comprise flowing a fluid comprising a compound (e.g., a crystallizable compound) over a substrate such that the compound crystallizes on the substrate. In certain embodiments, the devices and methods comprise separating a compound (e.g., a crystallizable compound, a solidifiable compound) from a solution. In some embodiments, the compound (e.g., the crystallizable compound, the solidifiable compound) can be recovered (e.g., at a high purity in solution). In certain embodiments, the substrate is orientated substantially vertically (e.g., such that flow of the fluid is driven by gravity). In some cases, the substrate comprises a plurality of crystallization-promoting structures. In certain embodiments, the substrate comprises flow redistributors (e.g., micropitches).

The use of devices and methods described herein offer several advantages as compared to traditional crystallization methods, including substantially eliminating the need for additional filtration steps to obtain a relatively high purity product (e.g., removing impurities from the surface of the crystal (e.g., as the crystal is forming on the surface), elimination of the need of slurry handling as generally no particles are generated in solution, constraining of the growth of crystals to a surface, increased reliability for manufacturing a product that meets purity and yield requirements, and replacing filtration and drying with a simple and relatively fast process of dissolution (e.g., reducing the number of unit operations as compared to traditional crystallization methods). In addition, alternative crystallizers such as falling film melt crystallizers generally require high concentrations of host material molecules in a melt (e.g., the melt having a temperature higher than the melting temperature of the host material). As such, the devices and methods described herein offer numerous additional advantages over traditional crystallizers, including falling film melt crystallizers, such as growing crystals at a temperature lower than the melting point enabling the crystallization and/or purification of temperature sensitive chemicals, reducing the energy cost of crystallization, reducing and/or eliminating the formation of impurities (e.g., preventing secondary chemical reactions the generate additional impurities), and/or reducing the concentration of host material molecules needed to form crystals.

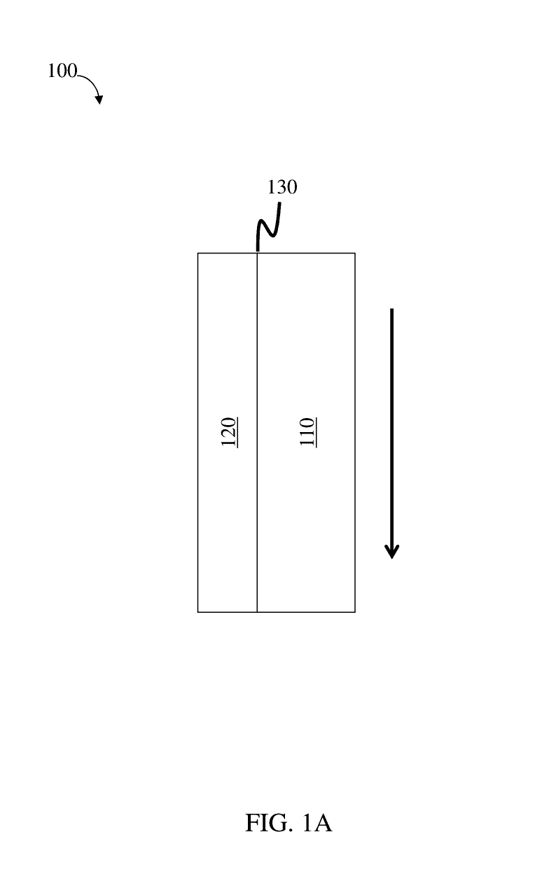

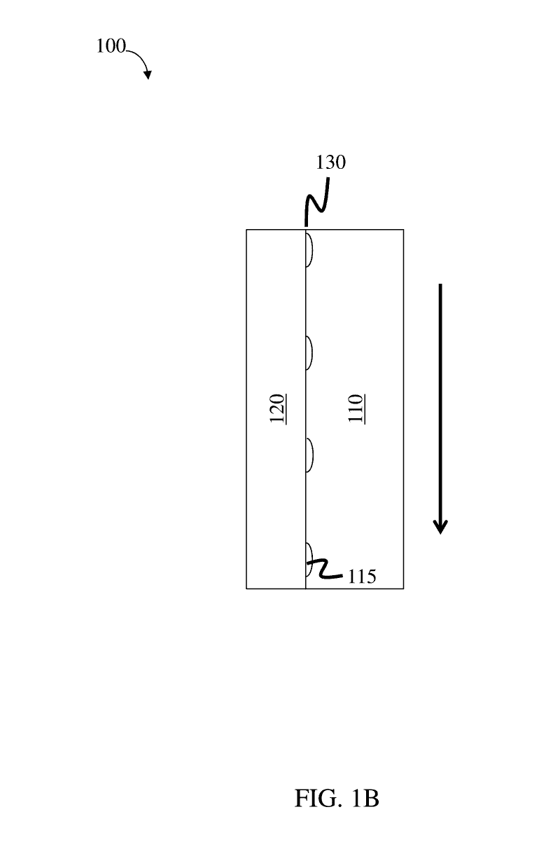

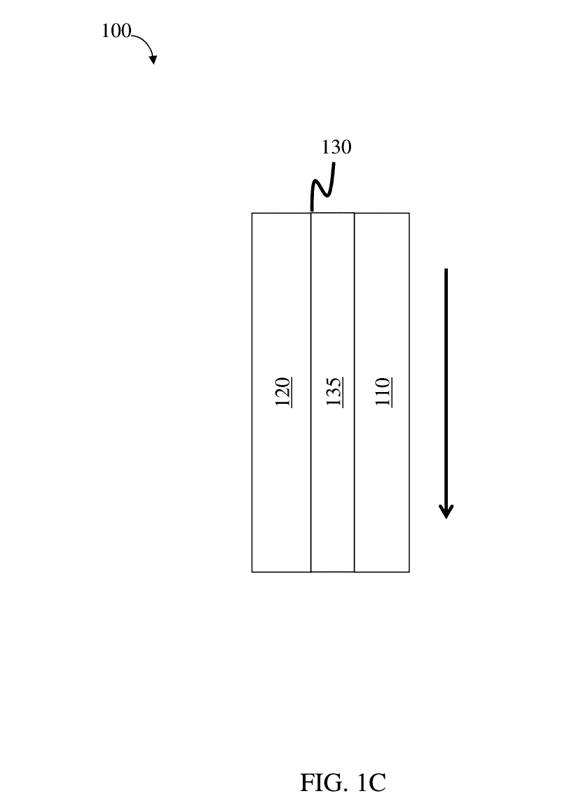

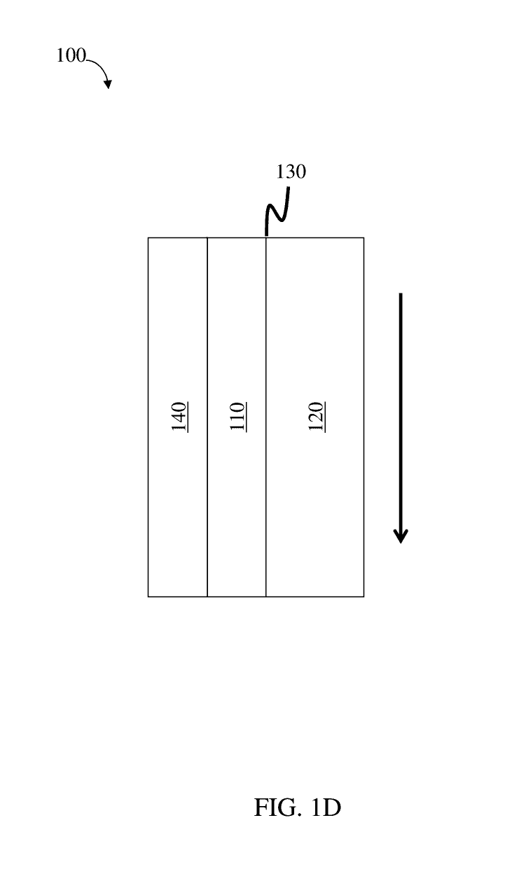

In some embodiments, the device is a falling film solution crystallizer. As illustrated in FIG. 1A, in some embodiments, device 100 (e.g., a device for crystallizing a compound) comprises a fluid 110 associated with a substrate 120 at an interface 130. Fluid 110 generally flows along surface 120 in the direction of the arrow, illustrated in FIG. 1A.

In some embodiments, the fluid comprises a crystallizable compound. The term “crystallizable” is known in the art and generally refers to a compound capable of forming crystals (e.g., a homogeneous substance with atoms arranged in a geometrical symmetric structure). In general, a wide variety of crystallizable compounds may be crystallized using the methods, described herein. In some embodiments, the crystallizable compound is a molecular species used in consumer products, such as pharmaceuticals, cosmetics, and/or food products. In some embodiments, the crystallizable compound is a small molecule (e.g., organic), inorganic salt, a macromolecule, biomolecules (e.g., protein, enzyme), and/or combinations thereof.

In some cases, the fluid comprises a solidifiable compound. The term “solidifiable” is known in the art and generally refers to a compound capable of precipitating (e.g., onto a surface) from a solution (e.g., a fluid comprising the solidifiable compound). In some such embodiments, the compound is capable of forming a solid layer on the substrate.

In some embodiments, the compound (e.g., the crystallizable compound, the solidifable compound) is a pharmaceutical compound such as an active pharmaceutical ingredient (e.g., drugs and/or drug precursors). As used herein, the term “active pharmaceutical ingredient” (also referred to as a “drug”) refers to an agent that is administered to a subject to treat a disease, disorder, or other clinically recognized condition, or for prophylactic purposes, and has a clinically significant effect on the body of the subject to treat and/or prevent the disease, disorder, or condition. Active pharmaceutical ingredients include, without limitation, agents listed in the United States Pharmacopeia (USP), Goodman and Gilman's The Pharmacological Basis of Therapeutics, 10th edition, McGraw Hill, 2001; Katzung, B. (editor), Basic and Clinical Pharmacology, McGraw-Hill/Appleton & Lange, 8th edition (Sep. 21, 2000); Physician's Desk Reference (Thomson Publishing); and/or The Merck Manual of Diagnosis and Therapy, 17th edition (1999), or the 18th edition (2006) following its publication, Mark H. Beers and Robert Berkow (editors), Merck Publishing Group, or, in the case of animals, The Merck Veterinary Manual, 9th edition, Kahn, C. A. (ed.), Merck Publishing Group, 2005. Preferably, though not necessarily, the active pharmaceutical ingredient is one that has already been deemed safe and effective for use in humans or animals by the appropriate governmental agency or regulatory body. For example, drugs approved for human use are listed by the FDA under 21 C.F.R. §§330.5, 331 through 361, and 440 through 460, incorporated herein by reference; drugs for veterinary use are listed by the FDA under 21 C.F.R. §§500 through 589, incorporated herein by reference. All listed drugs are considered acceptable for use in accordance with the present invention.

In certain embodiments, the active pharmaceutical ingredient is a small molecule. Exemplary active pharmaceutical ingredients include, but are not limited to, anti-cancer agents, antibiotics, anti-viral agents, anesthetics, anti-coagulants, inhibitors of an enzyme, steroidal agents, steroidal or non-steroidal anti-inflammatory agents, antihistamine, immunosuppressant agents, antigens, vaccines, antibodies, decongestant, sedatives, opioids, pain-relieving agents, analgesics, anti-pyretics, hormones, prostaglandins, etc.

Non-limiting examples of active pharmaceutical ingredients include ibuprofen, acetaminophen, and fenofibrate. Those of ordinary skill in the art, given the present disclosure, would be capable of applying the synthesis methods and systems described herein to other pharmaceutical active ingredients.

The fluid comprising the compound (the crystallizable compound, the solidifiable compound) is generally a solution. That is to say, in some embodiments, the fluid comprises a crystallizable compound and a solvent. In certain embodiments, the fluid comprises a solidifiable compound and a solvent. Non-limiting examples of suitable solvents include water, alcohols (e.g., methanol, ethanol, isopropanol, butanol), acetates (e.g., ethyl acetate), acetone, acrylonitrile, alkanes (e.g., peptane, hexane, butane, pentane, heptane, octane), and combinations thereof.

In some embodiments, the substrate is oriented non-horizontally (e.g., such that the fluid flow rate is substantially controlled by the orientation of the substrate). That is to say, in some embodiments, the substrate is oriented, relative to a substantially horizontal plane, at an angle of at least 10 degrees. In certain embodiments, the substrate is oriented at an angle of at least about 20 degrees, at least about 30 degrees, at least about 40 degrees, at least about 60 degrees, or at least about 80 degrees. In some cases, the substrate is oriented substantially vertically (e.g., at an angle of about 90 degrees relative to a horizontal plane). The substrate is generally oriented non-horizontally such that fluid flow along the substrate is driven substantially by gravitational forces. That is to say, in some cases, fluid can flow along the substrate without the need of an external pump and/or other fluid flowing devices. In a particular embodiment, the substrate is oriented substantially vertically such that fluid flows along a surface of the substrate due to the forces of gravity.

In some embodiments, the fluid may have an average flow rate. For example, in some embodiments (e.g., for a device of a bench-top size scale), the average flow rate of the fluid may be at least about 5 mL per minute, at least about 10 mL per minute, at least about 20 mL per minute, at least about 30 mL per minute, or at least about 40 mL per minute. In certain embodiments, the average flow rate of the fluid may be less than or equal to about 40 mL per minute, less than or equal to about 30 mL per minute, less than or equal to about 25 mL per minute, listening to about 20 mL per minute, or less than or equal to about 10 mL per minute. Combinations of the above-reference ranges are also possible (e.g., between about 5 mL per minute and about 40 mL per minute, between about 5 mL per minute and about 10 mL per minute, between about 10 mL per minute and about 30 mL per minute, between about 20 mL per minute and about 40 mL per minute). Other average flow rates are also possible. Those skilled in the art would be capable of selecting appropriate flow rates for the fluid based upon the teachings of the present disclosure for devices of relatively large size (e.g., scaled up devices). Those skilled in the art would be capable of selecting suitable methods for measuring the average flow rate including, for example, determining the flow rate of the fluid at the surface of the fluid not associated with the interface.

The substrate may comprise any suitable material. In some embodiments, the substrate comprises a material with a relatively high thermal conductivity (e.g., such that the temperature of the fluid flowing along the substrate may be controlled and/or modified). In some embodiments, the substrate comprises a metal (e.g., steel, aluminum, titanium, or the like). In some cases, the substrate may be substantially planar (i.e. the surface of the substrate at which the fluid interfaces with the substrate is substantially flat). In certain embodiments, the substrate may be a pipe (e.g., a tube, a cylinder, or the like). In a particular embodiment, the substrate is a hollow pipe. Those skilled in the art would understand that a cross-section of the pipe may not necessarily be substantially round and may have any suitable shape (e.g., rectangular, square, polygonal, triangular, circular, oval, irregularly shaped).

In some embodiments, the substrate comprises a plurality of crystallization promoting structures. In some embodiments, the surface of the substrate (e.g., the surface of the substrate associated with the fluid comprising the crystallizable compound) is relatively rough. That is to say, in certain embodiments, the surface of the substrate may comprise a plurality of structures and/or features on the order of microns such that the surface of the substrate is relatively rough. In some such embodiments, the plurality of crystallization promoting structures generally comprise such structures and/or features of the substrate. In some embodiments, the crystallization promoting structures (e.g., features and/or structures on the surface of the substrate) have an average height of at least about 1 micron, at least about 5 microns, at least about 10 microns, at least about 20 microns, or at least about 50 microns. In certain embodiments, the crystallization promoting structures have an average height of less than or equal to about 100 microns, less than or equal to about 50 microns, less than or equal to about 20 microns, less than or equal to about 10 microns, or less than or equal to about 5 microns. Combinations of the above-referenced ranges are also possible (e.g., between about 1 micron and about 10 microns, between about 1 micron and about 100 microns, between about 10 microns and about 100 microns). Other ranges are also possible.

The term “features” generally refers to a plurality of structures on the surface of a substrate (e.g., created by etching of the substrate, sandblasting of the surface, or deposition of a material such as a polymer on a surface of the substrate) with an average height on the order of microns. Such features may serve as, for example, nucleation sites for flowing fluids comprising a crystallizable compound over the features, such that a crystal layer forms on at least a portion of the surface of the substrate. In some embodiments, the surface of the substrate is sufficiently rough such that crystallization promoting structures are present on the surface of the substrate (e.g., the plurality of crystallization promoting structures are formed on a surface of the substrate by sand blasting the surface of the substrate such that the surface is relatively rough). In some cases, the crystallization promoting structures comprise the same material as the substrate, which are formed by etching the surface (e.g., sand-blasting the surface). In certain embodiments, crystallization promoting structures comprise a different material than the substrate (e.g., deposited on the surface of the substrate). Those skilled in the art would be capable of selecting materials, based upon the teaching of the specification, for depositing on a surface of the substrate such that a crystallizable compound forms a crystal layer on the surface of the substrate and/or on the deposited material. As described above, in some cases, the liquid comprises a solidifiable material and the crystallization promoting structures described herein may promote the precipitation of the solidifiable material on the substrate.

In certain embodiments, crystallization promoting structures may be formed by coating a surface of the substrate with a fluid comprising a relative high concentration of the crystallizable compound and a solvent and subsequently evaporating the solvent, such that the crystallizable compound forms a first crystal layer on the substrate. The first crystal layer may be relatively rough and comprise a plurality of crystallization promoting structures. Such first crystal layers may serve as, for example, nucleation sites for flowing subsequent fluids comprising a crystallizable compound over the first crystal layer, such that a second crystal layer forms on a surface of the first crystal layer. One or more of the crystal layers may be recovered. In some embodiments, the first crystal layer and the second crystal layer comprise the same crystallizable compound. In certain embodiments, the first crystal layer and the second crystal layer comprise different crystallizable compounds.

In certain embodiments, crystallization promoting structures may be formed by coating a surface of the substrate with a fluid comprising a relatively high concentration of the solidifiable compound and a solvent and evaporating the solvent, such that the solidifiable compound forms a first precipitated solid layer on the substrate. The first precipitated solid layer may be relatively rough and comprise a plurality of crystallization promoting structures.

The plurality of crystallizing promoting structures may comprise any suitable material (e.g., a material capable of promoting the crystallization of a compound on the surface of the substrate). For example, in some embodiments, the crystallizing promoting structures comprise a coating material (e.g., a coating material deposited on the surface of the substrate). The coating may comprise any suitable material capable of promoting crystallization including, but not limited to, polymers (e.g., polymers such that the crystallizable compound forms a crystal when the crystallizable compound contacts the polymer). In some cases, the coating material may be substantially smooth. In some embodiments, the coating material is substantially rough (e.g., having features and/or structures on the order of microns).

In some embodiments, the substrate comprises one or more flow redistributors. For example, at relatively low flow rates (e.g., less than about 5 mL per minute), flow redistributors may promote the distribution of the fluid along the surface of the substrate. In some embodiments, the flow redistributor is a coating (e.g., a polymer such as a hydrophilic polymer) such that the fluid flows along the surface of the substrate. In some embodiments, the flow redistributors comprise micropitches. In some embodiments, the micropitches are formed on the surface of the substrate. For example, in some such embodiments, the surface of the substrate may be threaded (e.g., augur shaped, screw threaded). In certain embodiments, the micropitches may comprise grooves (e.g., grooves in the surface of the substrate), microgrooves, and/or microstructures. The micropitches may be formed by any suitable means including, but not limited to, microfabrication. In certain embodiments, the flow redistributor comprises a ring (e.g., a polymer ring such as a nylon ring). In an exemplary embodiment, the flow redistributor is a nylon ring (e.g., having a thickness of about 0.38 mm). In certain embodiments, a plurality of flow redistributors (e.g., nylon rings) are spaced apart along the surface of the substrate by a particular distance (e.g., to provide local redistribution for the flow and/or to improve mixing of the fluid). In some such embodiments, the flow redistributors are separated by a distance of about 0.1 cm, about 0.5 cm, about 1 cm, or about 2 cm. Other spacings are also possible.

For example, as illustrated in FIG. 1B, device 100 comprises a plurality of flow redistributors 115 (e.g., micropitches) associated with substrate 120. In some such embodiments, interface 130 may be defined by the surface of substrate 120 and/or the surface of the plurality of flow redistributors 115 associated with fluid 110.

In a particular embodiment, the device comprises a substrate comprising a pipe (e.g., a steel pipe), a plurality of crystallization promoting structures associated with the substrate, (e.g., roughened and/or sand-blasted surface of the substrate) and one or more flow redistributors comprising micropitches (e.g., nylon rings) associated with the substrate.

In some embodiments, the temperature of the substrate and/or the temperature of the fluid may be controlled. In some embodiments, the temperature of the substrate is controlled such that the fluid comprising the crystallizable compound forms a crystal layer on at least a portion of the surface of the substrate. In some such embodiments, as illustrated in FIG. 1C, crystal layer 135 may form at interface 130 between surface 120 and fluid 110. While crystallization promoting structures are not shown in FIG. 1C, those skilled in the art would be capable of understanding that the crystal layer may form on at least a portion of a surface of the substrate and/or at least a portion of a surface the crystallization promoting structures (e.g., as shown in FIG. 1B).

The temperature of the interface surface of the substrate (i.e., the surface of the substrate at the interface between the substrate and the fluid) is generally less than a crystallization temperature of the crystallizable compound. In some embodiments, the fluid may have a particular average temperature greater than the crystallization temperature of the crystallizable compound, such that when the fluid contacts the substrate having an average temperature less than the crystallization temperature of the crystallizable compound, crystals of the crystallizable compound form at the interface between the substrate and the fluid comprising a crystallizable compound. Those skilled in the art would be capable of selecting suitable methods for determining the crystallization temperature of a crystallizable compound.

In some cases, the fluid comprising the crystallizable compound has a first average temperature that is less than the melt temperature of the crystallizable compound and greater than the crystallization temperature of the crystallizable compound. In some such embodiments, the substrate has a second average temperature less than the first average temperature and less than the crystallization temperature of the crystallizable compound, such that the crystal layer forms at the interface between the substrate and the fluid. For example, in certain embodiments, the average temperature of the substrate may be at least about −30° C., at least about −20° C., at least about −10° C., at least about 0° C., at least about 10° C. In some embodiments, the average temperature of the substrate may be less than or equal to about 20° C., less than or equal to about 10° C., less than or equal to about 0° C., less than or equal to about −10° C., or less than or equal to about −20° C. Combinations of the above-referenced temperatures may also be possible (e.g., between about −30° C. and about 10° C., between about −30° C. and about −10° C., between about −20° C. and about 0° C., between about −10° C. and about 10° C.). Other temperatures may also be possible.

In certain embodiments, the fluid may have a particular average temperature. Those skilled in the art would be capable of selecting an appropriate temperature for the fluid based upon the teachings of the present disclosure. For example, the temperature range may depend on the solvent selected. In some cases, the average temperature of the fluid ranges between the freezing temperature of the solvent and the boiling point or ignition point of the solvent. In some embodiments, the temperature of the substrate may be controlled by a temperature controlling layer. In certain embodiments, as illustrated in FIG. 1D, device 100 comprises a temperature controlling layer 140 associated with a second surface of substrate 110. In some embodiments, temperature controlling layer 140 comprises a temperature controlling device (e.g., a heater, a refrigeration unit, or the like). In a particular embodiment, temperature controlling layer 140 comprises a temperature controlling fluid. In some embodiments, the temperature controlling fluid comprises a coolant. Non-limiting examples of suitable coolants include water, antifreezing agents (e.g., ethylene glycol), and combinations thereof.

In some embodiments, the fluid comprising the crystallizable compound and the temperature controlling fluid flow substantially simultaneously. That is to say, in certain embodiments, the fluid comprising the crystallizable compound flows over a first portion of the substrate and the coolant flows over a second portion of the substrate, substantially simultaneously.

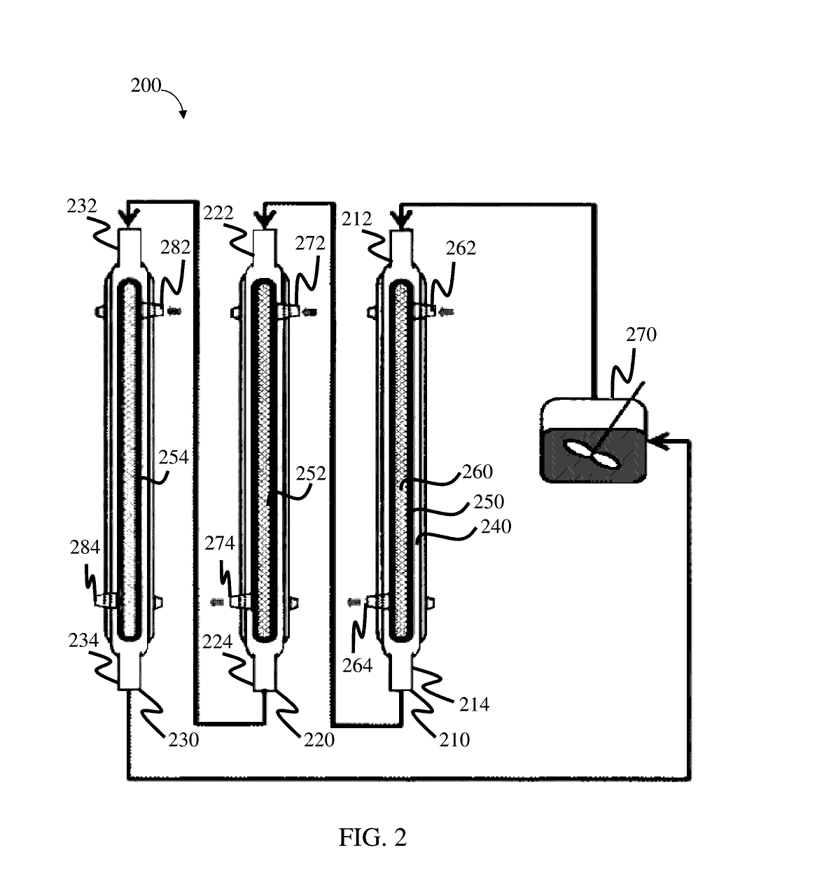

In some embodiments, one or more devices may be operated substantially simultaneously. In certain embodiments, two or more, three or more, or four or more devices may be used. In a particular embodiment, multiple devices are operated in series, for further purification, multiple stages of the crystallizer may be used in a sequence to crystallize and purify the dissolved deposited crystals from previous stages. For example, as illustrated in FIG. 2, system 200 comprises devices 210, 220, and 230. As an exemplary device, device 210 comprises fluid 240 (e.g., a fluid comprising a crystallizable compound), substrate 250, temperature controlling fluid 260, coolant inlet 262, and coolant outlet 264. In some such embodiments, fluid 240 enters device 210 at inlet 212, forming a crystal layer on substrate 250, and remaining fluid exits at outlet 214. In certain embodiments, outlet 214 is fluidically connected to inlet 222 of device 220. In some embodiments, outlet 224 is fluidically connected to inlet 232 of device 230 (further comprising outlet 234). In some such embodiments, outlet 214, 224, and/or 234 may be fluidically connected to a mixer 270 and/or inlet 212. In some embodiments, device 220 comprises coolant inlet 272 (e.g., coolant inlet 272 fluidically connected to coolant outlet 264), coolant outlet 274, and substrate 252 and device 230 comprises coolant inlet 282 (e.g., coolant inlet 282 fluidically connected to coolant outlet 274), coolant outlet 284, and substrate 254, such that fluid 240 may flow along a surface of substrates 252 and/or 254, forming a crystal layer on said substrates. In some embodiments, the parallel operation of the one or more devices may be repeated (e.g., in multiple stage operations) and may incorporate mixer 270. The use of multiple such falling film crystallizers and recycles may offer several advantages over the use of other traditional crystallizers including increased yield and purity of the crystallizable compound.

In some cases, after forming crystals or solids on one or more substrates, the compound can be recovered (e.g., for separation and/or purification). In some embodiments, the crystal layer and/or solid layer may be dissolved in a solvent (e.g., fresh warm solvent) and, optionally, pumped to a second unit where an additional crystallization can take place. In some such embodiments, the solvent comprising the compound (e.g., the crystallizable compound, the solidifiable compound) may have a higher purity of the compound as compared to the fluid comprising the compound prior to flowing the fluid. Non-limiting examples of suitable solvents for recovering the compound may include water, alcohols, acetates, acetone, acrylonitrile, or alkanes, as described above. In some embodiments, a solvent is flowed over the crystal layer such that the crystal layer dissolves in the solvent. In certain embodiments, the solvent is flowed over the solid layer such that the precipitated solid dissolves in the solvent. The term “dissolve” is given its meaning in the art and generally refers to the incorporation of a solid into a liquid such that a solution is formed.

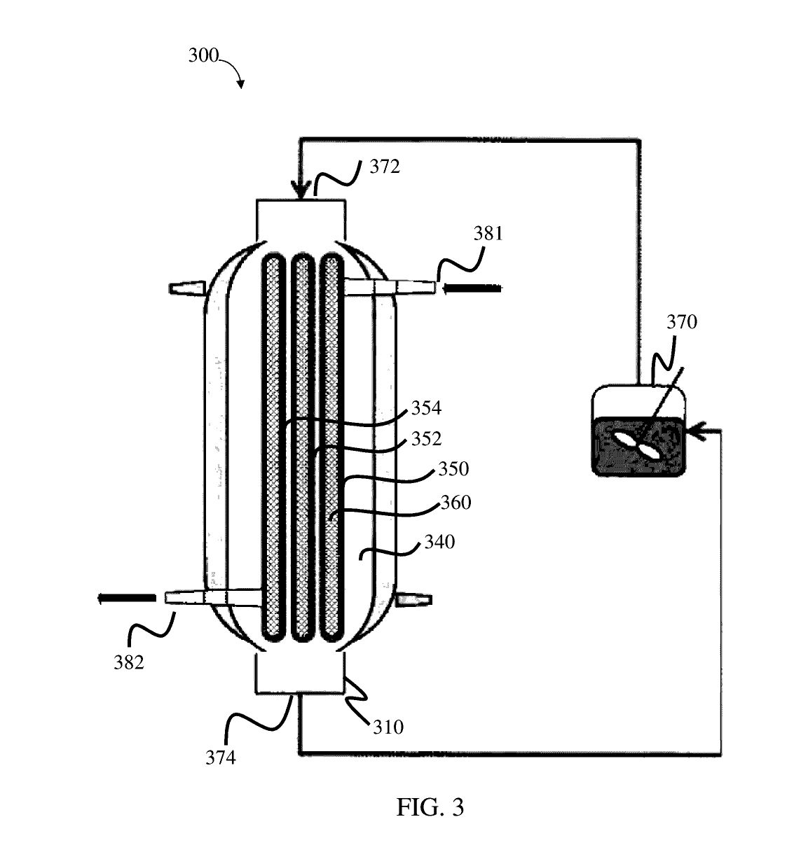

In some embodiments, parallel operation comprises flowing the fluid comprising the crystallizable compounds over one or more substrates (e.g., to increase the throughput). In certain embodiments, the device comprises two or more substrate, three or more substrates, or four or more substrates. For example, as illustrated in FIG. 3, system 300 comprises device 310 comprising a fluid comprising a crystallizable compound 340, substrates 350, 352, and 354, and temperature controlling fluid 360. Temperature controlling fluid 360 enters device 310 at inlet 372 and exits at outlet 374. In some embodiments, the temperature controlling fluid may be reused (e.g., inlet 381 and outlet 382 may be fluidically connected). In some embodiments, the parallel operation of the one or more substrates may be repeated (e.g., in multiple stage operations) and may incorporate mixer 370. In some such embodiments, the crystal layer may form on the one or more substrates substantially simultaneously.

In some embodiments, evaporation of the solvent from the surface of the falling fluid (e.g., by purging nitrogen over the falling fluid) may increase the concentration and enhance the driving force (i.e., the difference between the saturated concentration and the actual concentration at specific temperatures). In certain embodiments, an anti-solvent may be added (e.g., to change the solubility of the active pharmaceutical ingredients (APIs) and accelerate the crystal layer deposition over the substrate). Non-limiting examples of suitable anti-solvents include solvents (e.g., water, alcohol, acetate, acetone, acrylonitrile, alkane) as described herein.

As used herein, a “fluid” is given its ordinary meaning, i.e., a liquid or a gas. A fluid cannot maintain a defined shape and will flow during an observable time frame to fill the container in which it is put. Thus, the fluid may have any suitable viscosity that permits flow. If two or more fluids are present, each fluid may be independently selected among essentially any fluids (liquids, gases, and the like) by those of ordinary skill in the art.

The following examples illustrate embodiments of certain aspects of the invention.

### Example 1

The following examples demonstrate the purification of various pharmaceutical compounds using devices as described herein.

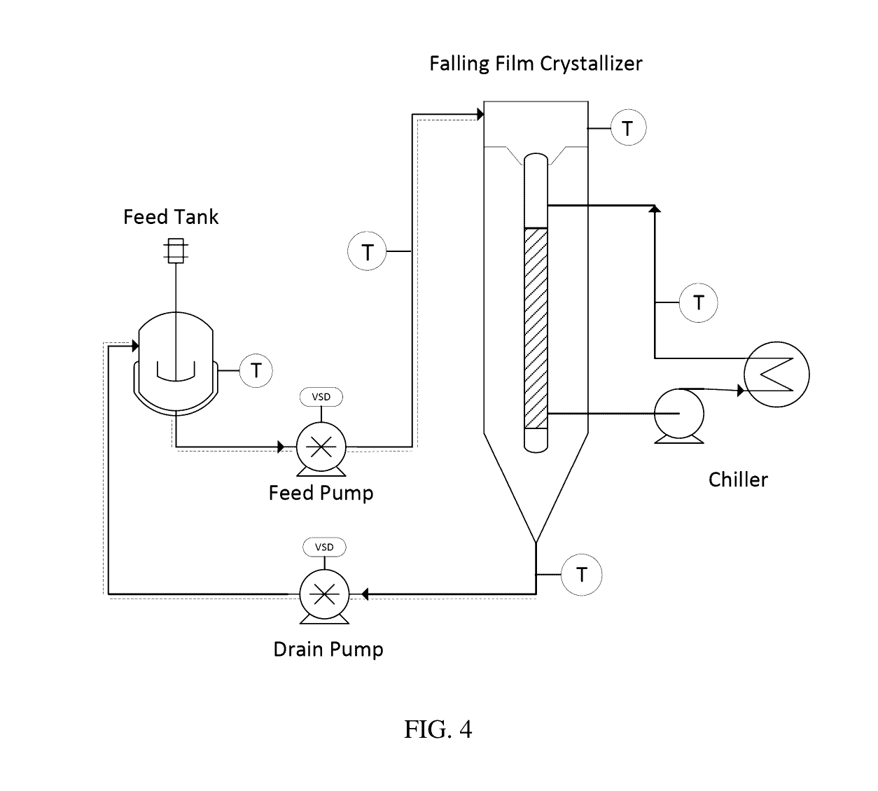

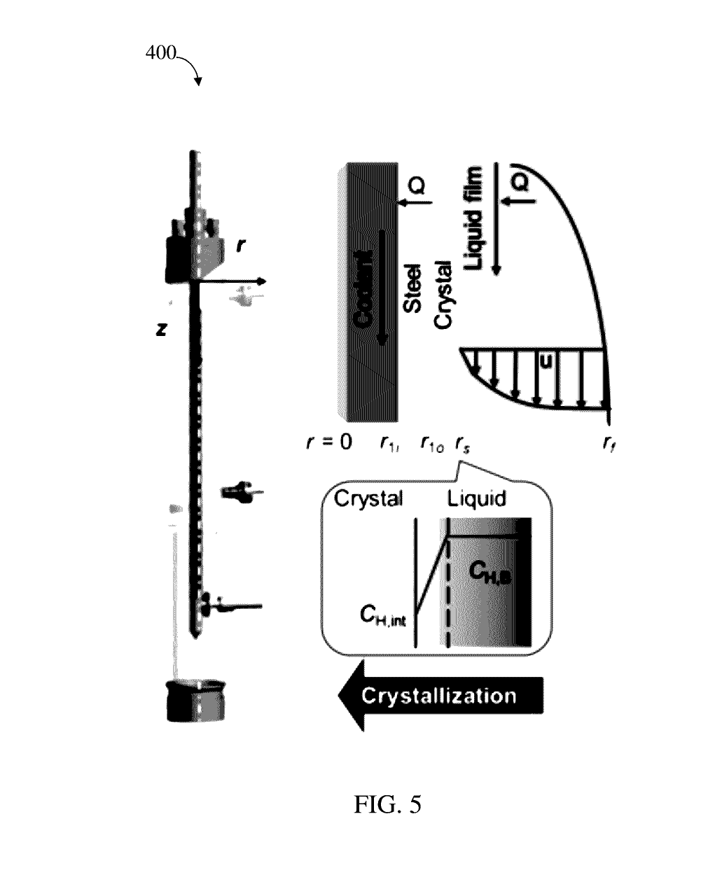

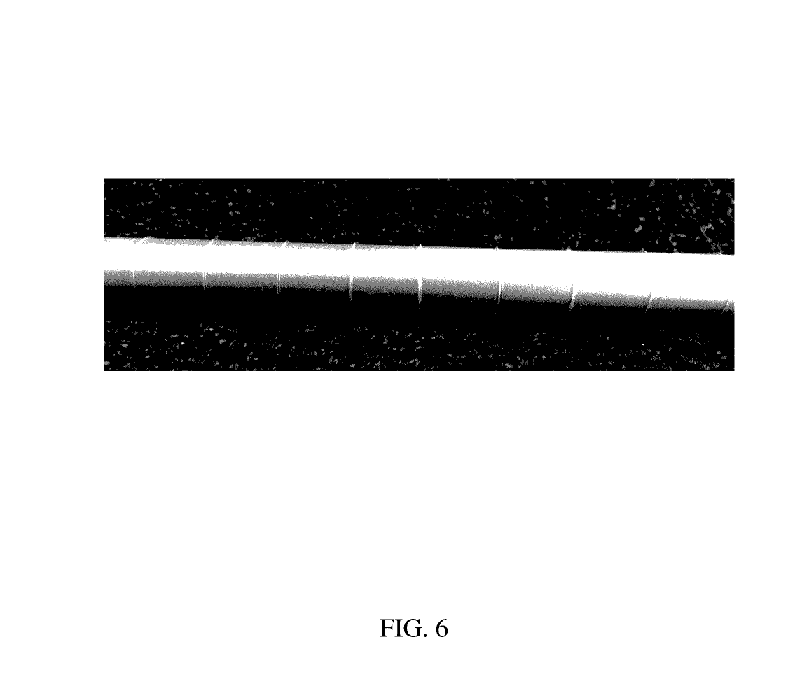

FIG. 4 shows a process flow diagram of the falling film crystallizer in a recycle loop used in the following examples. FIG. 5 shows a scheme of the column for an internally cooled tube surrounded by a falling liquid film. The liquid containing solute, impurities, and solvent entered at the top and slides down the outside wall of the core tube as a result of gravity. The core tube was cooled through coolant flowing inside the tube and the heat of the falling film, and the latent heat of crystallization are taken away from the core. The dramatic cooling of the film solution resulted in a supersaturated condition, which lead to depositing crystals on the surface of the core and a crystal layer grows at the interface of stainless steel wall (see FIG. 6) and falling film. The solution flowed into a temperature-controlled vessel and the solution was fed back into the falling film crystallizer through a peristaltic pump. The flow-rate of the solution, temperature of the feed, and temperature of the cold column were controlled over a range of processing conditions, as outlined below. FIG. 6 shows the surface of the core, which was sand-blasted and equipped with crystallization promoting structures, as well as flow redistributors (e.g., nylon rings). The core is a 40 cm long stainless steel 304 tube with the diameter of 127 mm and thickness of 16 mm. The flow redistributors are Nylon rings with 0.38 mm thickness, which are placed in 1 cm distance parallel to provide local redistribution for the flow and improve mixing of the film for the initial stages of the experiments. The surface of the core was sand-blasted with 500 micron glass beads to make the roughness which generally helps increase nucleation on the surface and keep the deposited layer of the crystal intact to the core.

The falling film column with a recycled loop was employed for the crystallization and purification of three saturated solutions of:

1) Acetaminophen (Sigma-Aldrich) and a mixture of ethanol (Koptec, 200 proof) and deionized water with a volume ratio of 50:50 at an initial temperature of 65° C. Metacetamol (Sigma-Aldrich) was manually added into the initial solution as impurity with 5% mass ratio to the amount of the Acetaminophen in the solution to make the initial purity of 95% for the feed solution.

2) Fenofibrate (Xian Shunyi Bio-Chemical Technology Co., Ltd.) dissolved in a mixture of ethanol (Koptec, 200 proof) and Ethyl Acetate (BDH Chemicals) with a volume ratio of 30:70 at an initial temperature of 65° C. Fenofibric Acid (Xian Shunyi Bio-Chemical Technology Co., Ltd.) was manually added into the initial solution as impurity with 2% mass ratio to the amount of the Fenofibrate in the solution to make the initial purity of 98% for the feed solution.

3) Fenofibrate (Xian Shunyi Bio-Chemical Technology Co., Ltd.) dissolved in ethanol (Koptec, 200 proof) at an initial temperature of 65° C. Fenofibric Acid (Xian Shunyi Bio-Chemical Technology Co., Ltd.) was manually added into the initial solution as impurity with 2% mass ratio to the amount of the Fenofibrate in the solution to make the initial purity of 98% for the feed solution.

Table 1 summarizes the solutions for each system described above.

TABLE 1
The crystallization systems for the falling film experiments.

| | Main Compound | Impurity | | | |
|---|---|---|---|---|---|
| | (Initial | (Initial | | | |
| | Concentration, W | Concentration, W | | Growth | Distribution |
| Systems | %) | %) | Solvent | Rate | Coefficient |
| System I | Acetaminophen | Metacetamol | Ethanol & Water | Slow | High |
| | (95%) | (5%) | (50:50 V %) | | |
| System | Fenofibrate | Fenofibric Acid | Ethyl Acetate & | High | Low |
| II | (98%) | (2%) | Ethanol | | |
| | | | (70:30 V %) | | |
| System | Fenofibrate | Fenofibric Acid | Ethanol | Slow | Low |
| III | (98%) | (2%) | (100%) | | |

The temperature controlling (cooling) liquid in core was a mixture of 30% ethylene glycol and 70% water in mass and its flow rate was 24 L/min. The falling film solution was recirculated via a peristaltic pump, which transfers solution from a stirred tank of 200 mL in the recycle loop. Samples of solution were taken at the stirred tank and at the bottom of the column to determine the concentration of the APIs and related impurities with high-performance liquid chromatography (Agilent 1200).

Table 2 shows the yield and purity of System I (Acetaminophen from Ethanol:Water) from the falling film experiments with a range of flow-rates and cooling temperatures from an initial purity of 95%.

TABLE 2
Yield and purity of System I (Acetaminophen from Ethanol:Water) from
the falling film experiments.

| | Yield (%) | | Purity (%) | |
|---|---|---|---|---|
| Cooling Temperature | 0 | 10 | 0 | 10 |
| 5 mL/min flow-rate | 71 ± 0.5 | 66 ± 0.4 | 96.6 ± 0.2 | 97.0 ± 0.2 |
| 20 mL/min flow-rate | 69 ± 0.6 | 65 ± 0.4 | 96.8 ± 0.2 | 97.4 ± 0.25 |
| 30 mL/min flow-rate | 68 | | 97.1 | |

Table 3 shows the yield and purity of System II (Fenofibrate from Ethyl Acetate:Ethanol) from the falling film experiments with a range of flow-rates and cooling temperatures from an initial purity of 95%.

TABLE 3
Yield and purity of System II (Fenofibrate from Ethyl Acetate:Ethanol)
from the falling film experiments.

| | Yield (%) | | Purity (%) | |
|---|---|---|---|---|
| Cooling Temperature | 0 | 10 | 0 | 10 |
| 5 mL/min flow-rate | 76 ± 0.5 | 71 ± 0.4 | 98.4 ± 0.3 | 98.3 ± 0.3 |
| 20 mL/min flow-rate | 74 ± 0.4 | 68 ± 0.4 | 98.8 ± 0.1 | 98.4 ± 0.2 |
| 40 mL/min flow-rate | 70 | | 98.9 | |

Table 4 shows the yield and purity of System III (Fenofibrate from Ethanol) from the falling film experiments with a range of flow-rates and cooling temperatures from an initial purity of 98%.

TABLE 4
Yield and purity of System III (Fenofibrate from Ethanol) from the falling
film experiments.

| | Yield (%) | | Purity (%) | |
|---|---|---|---|---|
| Cooling Temperature | 0 | 10 | 0 | 10 |
| 5 mL/min flow-rate | 74 ± 1.7 | 68 ± 2.2 | 99.2 ± 0.1 | 99.1 ± 0.1 |
| 10 mL/min flow-rate | 73 ± 1.4 | 69 ± 1.7 | 99.2 ± 0.1 | 99.3 ± 0.1 |
| 15 mL/min flow-rate | 72 | 69 ± 2.1 | 99.4 ± 0.1 | 99.4 ± 0.2 |

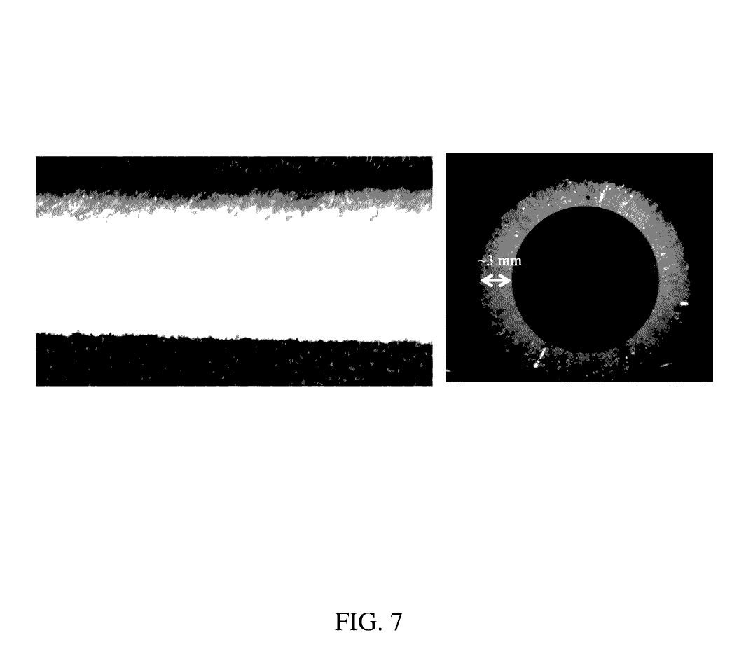

FIG. 7 shows the deposited crystal layer (e.g., from the acetaminophen system) on the substrate, where the crystals are relatively uniform and in fine size and the layer is relatively uniform and symmetrical.

### Example 2

The falling film column with a recycled loop was applied for the purification of ibuprofen (Xian Shunyi Bio-Chemical Technology Co. Ltd., pharmaceutical grade) from a mixture of ethanol (Koptec, 200 proof) and water (Sigma Aldrich, CHROMASOLV®Plus) with the mass concentration ratio of 80:20 at an initial temperature of 62° C. Ketoprofen was manually added into the initial solution as impurity. The temperature controlling (cooling) liquid in the core was a mixture of 30% ethylene glycol and 70% water in mass and its flow rate was 24 L per minute. The falling film was recirculated via peristaltic pump which drew solution from a stirred tank of 200 mL in the recycle loop. Samples of solution were drawn at the stirred tank to determine the concentration with high-performance liquid chromatography (Agilent 1200) of ibuprofen and ketoprofen.

The yield of ibuprofen from the falling film experiment is 67.23% in the purity of ibuprofen is improved to 97.40% from an initial purity of 95.23%.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape, orientation, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, circular/circle, rectangular/rectangle, triangular/triangle, cylindrical/cylinder, elliptical/ellipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, east, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described.

What is claimed:

1. A method for obtaining a crystallized compound, comprising:
flowing a fluid comprising the compound, the fluid having a first temperature less than the melt temperature of the compound, over at least a portion of a substrate having a second temperature less than the first temperature, such that the compound crystallizes in a crystal layer on at least a portion of the substrate, wherein the substrate is oriented substantially vertically; and
recovering the crystallized compound.

2. A method for separating a solidifiable compound, comprising:
flowing a fluid comprising the compound, the fluid having a first temperature less than the melt temperature of the compound, over at least a portion of a substrate having a second temperature less than the first temperature, such that the compound precipitates a solid on at least a portion of the substrate, wherein the substrate is oriented substantially vertically; and
recovering the precipitated solid.

3. A method as in claim 1, wherein recovering the crystallized compound comprises flowing a solvent over the crystal layer such that the crystal layer dissolves in the solvent.

4. A method as in claim 1, wherein recovering the precipitated solid comprises flowing a solvent over the crystal layer such that the precipitated solid dissolves in the solvent.

5. A method as in claim 1, wherein the method further comprises flowing a temperature controlling fluid over at least a second portion of the substrate.

6. A method as in claim 1, wherein the fluid comprising the compound has a flow rate of between about 5 mL/min and about 40 mL/min.

7. A method as in claim 1, wherein flowing the fluid does not comprise the use of a pump.

8. A method as in claim 1, wherein flowing the fluid comprises flowing the fluid vertically along the substrate.

9. A method as in claim 1, wherein the compound is a pharmaceutical compound.

10. A method as in claim 1, wherein the substrate comprises a plurality of crystallization promoting structures.

11. A method as in claim 1, wherein the plurality of crystallization promoting structures comprises features having an average height of at least about 1 micron and less than or equal to about 100 microns.

12. A method as in claim 1, wherein the substrate further comprises one or more flow redistributors.

13. A method as in claim 12, wherein the one or more flow redistributors comprises micropitches.

14. A method as in claim 1, wherein the substrate comprises a metal.

15. A method as in claim 1, wherein the fluid comprising the compound is a solution.

16. A device for crystallizing and/or separating a compound, comprising:
a substantially vertical substrate having a first portion adapted to receive a flowing fluid comprising a crystallizable compound, and a second portion adapted to receive a temperature controlling fluid; and
a plurality of crystallization-promoting structures on the first portion of the substrate.

17. A device as in claim 16, wherein the substrate comprises a metal.

18. A device as in claim 16, wherein the crystallizable compound is a pharmaceutical compound.

19. A device as in claim 16, wherein the temperature controlling fluid comprises ethylene glycol.

20. A device as in claim 16, wherein the plurality of crystallization promoting structures comprises features having an average height of at least about 1 micron and less than or equal to about 100 microns.

21. A device as in claim 16, wherein the device further comprises a flow redistributor.

22. A device as in claim 16 wherein the fluid comprising a crystallizable compound is a solution.

23. A device as in claim 16, wherein the fluid comprising a crystallizable compound comprises a solvent.
